(19) United States
(12) Patent Application Publication
(10) Pub. No.: US 2020/0079715 A1
(43) Pub. Date: Mar. 12, 2020

(54) 2-POSITION MODIFICATION FOR SYNTHESIS OF RESORCINOL SCAFFOLDING

(71) Applicant: Canopy Holdings, LLC, Littleton, CO (US)

(72) Inventors: Robert Davis, Longmont, CO (US); Jacob Black, New Haven, CT (US); Thomas Smeltzer, Longmont, CO (US); Sean Colvin, Gunbarrel, CO (US)

(21) Appl. No.: 16/563,673

(22) Filed: Sep. 6, 2019

Related U.S. Application Data
(60) Provisional application No. 62/727,951, filed on Sep. 6, 2018.

(51) Int. Cl. C07C 37/62

(52) U.S. Cl. CPC C07C 37/62

(57) ABSTRACT

A resorcinol with modifications at the 2-position is provided. The reactant resorcinol may have a variety of functional groups at each of the 1, 3, and 5 position such as a hydroxide, a lower alkyl group, a phenyl, a substituted phenyl, a lower alkenyl, or a lower alkynyl sp² carbon group (e.g., substituted phenyl, vinyl), sp (e.g., alkyne), hydrogen. The resorcinol is modified at the 2-position with a nucleophile or an electrophile. The resulting resorcinol may serve as a stable intermediate for the synthesis of cannabinoid or cannabinoid derivatives.

### BACKGROUND

While the medicinal value of various cannabinoids was anecdotally reported for thousands of years, it was not until the isolation of Δ⁹-tetrahydrocannabinol (Δ⁹-THC) in 1964 that cannabinoids came into the spotlight as the agents responsible for the pronounced physiological effects of cannabis. In an effort to identify the origin of Δ⁹-THC's effects, the human G-protein coupled receptors cannabinoid receptor 1 (CB1) and cannabinoid receptor 2 (CB2) were discovered, unearthing a complex signaling pathway within human physiology: the endocannabinoid system. This system is responsible for regulating numerous physiological processes, including memory, mood, metabolism, immune function, appetite, thermoregulation, sleep and analgesia.

CB1 and CB2 are activated by the mammalian-produced endocannabinoids anandamide (AEA) and 2-arachidonylglycerol (2-AG) or the C. sativa produced phytocannabinoid Δ⁹-THC. Functional evidence has suggested more cannabinoid receptor sub-types exist, and in recent years several candidates have been identified, namely, GPR55, GPR18, and GPR119. The role of GPR55 is still under investigation, but phenotypic evidence suggests it may play a role in pulmonary arterial hypertension. GPR55 also appears to mediate rhoA, cdc42, and racl activity, all important proteins in the cell cycle. Studies suggest that GPR18 is the receptor for N-arachidonoyl glycine (NAGly), a metabolite of AEA. Binding of NAGly to GPR18 initiates directed microglial migration in the central nervous system. GPR18 is also activated by Resolvin D2 (RvD2), which upon binding leads to the resolution of inflammatory responses and inflammatory disease states in animal models. GPR119 is found predominantly in the pancreas and gastrointestinal tract and has been shown to regulate insulin secretion. Activation of GPR119 has been shown to limit food intake as well as weight gain in rat models.

The proposed functions of these enzymes make them valuable targets for therapeutics and presents a need for tool compounds for their study. In order to study these receptors, cannabinoids and cannabinoid-like compounds that exhibit selectivity for these potential sub-types, but show no affinity for the traditional cannabinoid receptors (CB1 and CB2) are needed. While the naturally abundant Δ⁹-THC is well studied, and Δ⁹-cannabidiol (CBD) has recently gained attention, over 100 other minor cannabinoids are produced in relatively small quantities by the cannabis plant. Many of these minor cannabinoids have shown little to no affinity for CB1 or CB2, but nevertheless show notable biological responses.

Of particular interest are the cannabinoids cannabichromene (CBC), cannabigerol (CBG), and cannabinol (CBN), which have been anecdotally implicated in a variety of effects. This, correspondingly, has incited consumer demand and warranted further scientific exploration. Initial studies have revealed interaction of CBG with the potential receptor GPR55, α₂-adrenergic receptor, and 5-HT_{1A} receptor. CBC has been shown to interact with TRPV1 and TRPA1, while the biological profile of CBN is relatively unknown. Further, the CBD homologs Δ⁸-cannabidiol and cannabidivarin are known to have anticonvulsant properties but studies have been limited due to lack of available material. Meanwhile, the CBG homolog cannabigerivarin has greater binding affinity for GPR55 than CBG, but has otherwise gone largely unnoticed.

Due to limited availability of these compounds from natural sources, artificial synthesis of cannabinoids may provide a reliable and inexpensive source of such cannabinoids. Despite decades of effort in this area, current methods of production leave much to be desired. For example, current technology for the synthesis of cannabinoids is limited to certain cannabinoids. Additionally, these methods result in low yields of the desired cannabinoids, high levels of impurities, and/or the necessity to work with volatile and dangerous chemicals. Thus, the current technology to synthesize cannabinoids cannot practically be reproduced on a commercial scale.

As such, the exploration of the potential pharmaceutical and nutraceutical benefits of cannabinoids would benefit from technology that reduces costs, improves yields, reduces impurities, and increases safety when synthesizing cannabinoids.

It is with respect to these and other considerations that the technology is disclosed. Also, although relatively specific problems have been discussed, it should be understood that the embodiments presented should not be limited to solving the specific problems identified in the introduction.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

Aspects of the technology described herein provide for the synthesis of various cannabinoids, cannabinoid derivative, and synthetic intermediates useful in the synthesis of cannabinoids. For example, the technology described herein provides methods for modification of resorcinol groups at the 2-position to create stable intermediaries (scaffold or scaffolding) that may be used as a precursor for a cannabinoid of cannabinoid derivatives. One may use such modified 5 resorcinols as substrates for the synthesis of a variety of cannabinoids and cannabinoid derivatives and selected coupling partners for said synthesis.

Aspects of the technology relate to a compound having the following structure:

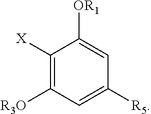

In aspects of the technology, X is selected from the group consisting of I, bis(pinacolato)diboron (Bpin), B(OH)₂, B(OR₆)₂, Br, Sn(R₇)₃, Si(Me)₃, Si(R₈)₃, OTf, Cl, Mg(II)I, Zn(II)I, cuprate, lithium, Mg(II)Br, and Zn(II)Br, each of R₁ and R₃ is selected from the group consisting of THP, Benzyl, and a silane protecting group, and R₅ is selected from the group consisting of a lower alkyl group, a phenyl, a substituted phenyl, a lower alkenyl, and a lower alkynyl.

In some aspects of the technology, in the compound above, R₁ and R₃ are different. In further aspects of the technology, R₆, R₇, and R₈, is selected from the group consisting of a lower alkyl group, a phenyl, a substituted phenyl, a lower alkenyl, and a lower alkynyl.

Further aspects of the technology further relate to a compound having the following structure:

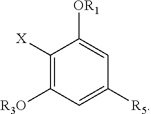

In aspects of the technology, X is selected from the group consisting of bis(pinacolato)diboron (Bpin), B(OH)₂, B(OR₆)₂, Br, Sn(R₇)₃, Si(Me)₃, Si(R₈)₃, OTf, Mg(II)I, Zn(II)I, a cuprate, lithium, Mg(II)Br, and Zn(II)Br, each of R₁ and R₃ is selected from the group consisting of hydrogen and acetate, and R₅ is a functional group selected from the group consisting of a lower alkyl group, a phenyl, a substituted phenyl, a lower alkenyl, and a lower alkynyl. In some aspects of the technology, R₁ and R₃ are different. In some aspects of the technology, each of R₆, R₇, and R₈, is selected from the group consisting of a lower alkyl group, a phenyl, a substituted phenyl, a lower alkenyl, and a lower alkynyl.

Further aspects of the technology relate to a compound having the following structure:

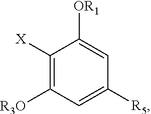

where X is selected from the group consisting of B(OR₆)₂, Sn(R₇)₃, Si(R₈)₃, OTf, Cl, Mg(II)I, Zn(II)I, a cuprate, and Zn(II)Br; each of R₁ and R₃ is selected from the group consisting of methyl and methoxymethyl (MOM); and R₅ is a functional group selected from the group consisting of a lower alkyl group, a phenyl, a substituted phenyl, a lower alkenyl, and a lower alkynyl. In aspects of the technology, R₁ and R₃ are different. In further aspects of the technology, of R₆, R₇, and R₈, is selected from the group consisting of a lower alkyl group, a phenyl, a substituted phenyl, a lower alkenyl, and a lower alkynyl.

Further aspects of the technology relate to a compound having the following structure:

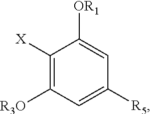

where X is selected from the group consisting of Bpin, B(OH)₂ and lithium, where R₁ and R₃ each is MOM, where R₅ is selected from the group consisting of a lower alkyl group, a phenyl, a substituted phenyl, a lower alkenyl, and a lower alkynyl. In further aspects of the technology, R₁ and R₃ are different.

Further aspects of the technology relate to a compound having the following structure:

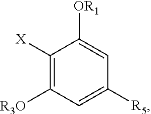

where X is selected from the group consisting of Bpin, B(OH)₂, Si(Me)₃, and lithium, and where each of R₁ and R₃ is MOM, and where R₅ is selected from the group consisting of a lower alkyl group, a phenyl, a substituted phenyl, a lower alkenyl, and a lower alkynyl.

Further aspects of the technology relate to a compound having the following structure:

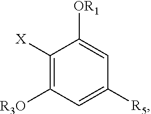

where X is selected from the group consisting of Mg(II)Br and a cuprate, where each of R₁ and R₃ is methyl; and where R₅ is selected from the group consisting of a lower alkyl group, a phenyl, a substituted phenyl, a lower alkenyl, and a lower alkynyl.

Further aspects of the technology relate to a compound having the following structure:

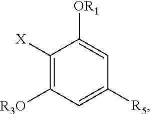

where X is Cl, where each of R₁ and R₃ is acetate; and where R₅ is selected from the group consisting of a lower alkyl group, a phenyl, a substituted phenyl, a lower alkenyl, and a lower alkynyl.

Further aspects of the technology relate to a method of halogenating a resorcinol. In aspects of the technology, the method includes providing a first compound having the following structure:

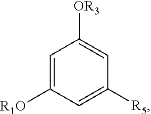

wherein R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and wherein R₅ is selected from the group consisting of a lower alkyl group, a phenyl, a substituted phenyl, a lower alkenyl, and a lower alkynyl. The method further includes, in aspects, treating the compound with a halogenating agent, wherein the halogenating agent is selected from the group consisting of bromine (Br₂), iodine (I₂), N-chlorosuccinimide (NCS), N-bromosuccinimide (NBS), N-iodosuccinimide (NIS), 1,3-dichloro-5,5-dimethylhydantoin (DCDMH), 1,3-dibromo-5,5-dimethylhydantoin (DBDMH), trichloroisocyanuric acid (TCICA), dibromoisocyanuric acid (DBICA), and tetrabutylammonium tribromide. Such treatment may be performed in the presence of a solvent.

In aspects of the technology, the method also includes adding a catalyst, wherein the catalyst is selected from the group consisting of hydrochloric acid, acetic acid, p-toluenesulfonic acid, trifluoroacetic acid, sodium bicarbonate, sodium hydroxide, an amine, and a combination thereof. In aspects of the technology, the solvent is selected from the group consisting of water, tetrahydrofuran, methanol, acetonitrile, methyl t-butyl ether and a combination thereof.

Aspects of the technology further relate to a method of modifying a resorcinol comprising. The method includes providing the resorcinol having the following structure:

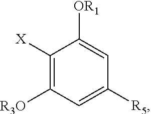

wherein x is a halogen, R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and R₅ is selected from the group consisting of a lower alkyl group, a phenyl, a substituted phenyl, a lower alkenyl, and a lower alkynyl. The method further includes treating the resorcinol with bis(pinacoloto)borane or hexabutylditin in the presence of a suitable catalyst, comprising palladium, nickel, copper, gold, silver, iron, or cobalt, Pd(ddpf)₂Cl₂, Pd(PPhl)₂Cl₂, Pd(PPh₃)₄, Ni(cod)₂, NiI₂, NiBr₂, NiCl₂, and Ni(acac)₂, or a combination thereof in the presence of a base selected from the group consisting of a pyridine, a bipyridine, a phenanthroline, a terpyridine, a bisoxazoline, pyridine bisoxazoline, a phosphine, a metal halide salt, a metal alkoxide salt, an amine, a carbonate, and a combination thereof. In some aspects of the technology, X is selected from the group consisting of chlorine, bromine, iodine, acetate, and triflate.

Aspects of the technology further relate to a method of modifying a resorcinol. The method includes providing a resorcinol having the following structure:

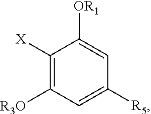

wherein X is a halogen or a metal and each of R₁ and R₃ is hydrogen. The method further comprises treating the resorcinol with a base selected from the group consisting of sodium bicarbonate, potassium carbonate, triethylamine, dimethylamino pyridine, and a combination thereof, in the presence of a solvent selected from the group consisting of DMF, THF, and dichloromethane; and treating the mixture with a halogenating agent selected from the group consisting of methyl iodide, benzyl bromide, trimethylsilyl chloride, t-butyldimethylsilyl chloride, SEM chloride, and acetyl chloride.

Aspects of the technology further relate to a method of modifying a resorcinol comprising. In aspects of the technology, the method includes providing the resorcinol having the following structure:

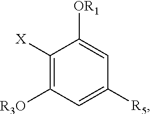

wherein x is a halogen; R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and wherein R₅ is selected from the group consisting of a lower alkyl group, a phenyl, a substituted phenyl, a lower alkenyl, and a lower alkynyl. The method further comprises treating the resorcinol with a metallating species to form a treated resorcinol. The method further comprises reacting the treated resorcinol with electrophilic metal species in the presence of a solvent. In some aspects of the technology, the method, the metallating species is selected from the group consisting of zinc, lower alkyllithium, and magnesium. In some aspects of the technology, the electrophilic metal species is selected from the group consisting of boronyl chlorides, stannyl chlorides, and silyl chlorides. In some aspects of the technology, the solvent is selected from the group consisting of dimethylformamide (DMF), dimethylacetamide, tetrahydrofuran (THF), toluene, dichloromethane, acetonitrile, dimethylsulfoxide, hydrocarbon solvents and a combination thereof.

Aspects of the technology further include a method of modifying a resorcinol. The method includes providing the resorcinol having the following structure:

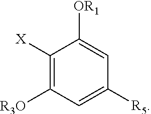

In aspects, x is a halogen, R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and R₅ is selected from the group consisting of a lower alkyl group, a phenyl, a substituted phenyl, a lower alkenyl, and a lower alkynyl. The method further includes treating the resorcinol with a di-metal species to form a treated resorcinol, and reacting the treated resorcinol with electrophilic metal species, in the presence of a solvent.

Aspects of the technology include where the di-metal species is selected from the group consisting of bis(pinacoloto)borane, hexabutylditin in the presence of a suitable catalyst, including palladium, nickel, copper, gold, silver, iron, or cobalt, Pd(ddpf)₂Cl₂, Pd(PPh₃)₂Cl₂, Pd(PPh₃)₄, Ni(cod)₂, NiI₂, NiBr₂, NiCl₂, and Ni(acac)₂. Aspects of the technology further include that the solvent is selected from the group consisting of dimethylformamide (DMF), dimethylacetamide, tetrahydrofuran (THF), toluene, dichloromethane, acetonitrile, dimethylsulfoxide, hydrocarbon solvents and a combination thereof.

### DETAILED DESCRIPTION

### I. Definitions

The terminology used in this disclosure is for the purpose of describing particular embodiments only and is not intended to be limiting of the disclosure. As used in the description of the embodiments of the disclosure and the appended claims, the singular forms “a,” “an,” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, amount, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs.

Halogenated resorcinols may serve as a stable synthetic intermediate that may be used for the synthesis of both known and unknown cannabinoids. As used herein, the term halogenated resorcinol refers not only to resorcinols that have a halogen as a functional group, but includes resorcinols with an electrophile, such as acetate or triflate, as a functional group.

### II. Addition at 2-Position of 5-Functionalized Resorcinols

Aspects of the technology include halogenating resorcinols. In particular, a resorcinol of the form:

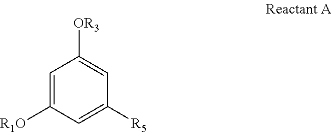

may be reacted with halides such as chloride (Cl+), bromide (Br+), iodide (I+), acetate (+OAc), and triflate (+OTf) to form a 2-halogniated resorcinol the compound:

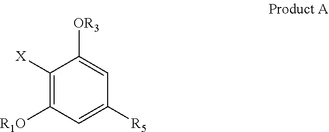

Thus, the proposed reaction is:

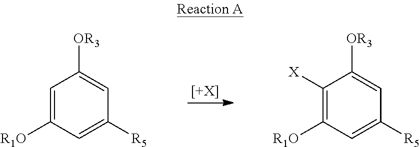

Resorcinols may be selected with particular functional groups at R1, R3, and R5 for applications. For instances, for the synthesis of certain cannabinoids (e.g., cannabidiol), a resorcinol may be selected with the desired functional group (e.g., n-pentyl at R5). In other instances, synthesis of certain cannabinoids and cannabinoid derivatives may include other intermediate steps where it may be desirous to have other functional groups at R1, R3, and R5.

As such, in Reaction A, X may be the halide described above. In aspects, R1 and R3 each may be one of H, acetate or other esters, methyl or other simple alkyl groups, benzyl or other ethers carbonates, a silane protecting group (e.g., a lower alkyl silane), or any other useful functional group. In aspects of the technology, R5 may be a lower alkyl group, a vinyl, a substituted vinyl, a phenyl, a substituted phenyl, a lower alkenyl, or a lower alkynyl group.

The halogenation described above may be accomplished by treatment of Reactant A with halogenating agents including but not limited to bromine (Br₂), iodine (I₂), N-chlorosuccinimide (NCS), N-bromosuccinimide (NBS), N-iodosuccinimide (NIS), 1,3-dichloro-5,5-dimethylhydantoin (DCDMH), 1,3-dibromo-5,5-dimethylhydantoin (DBDMH), trichloroisocyanuric acid (TCICA), dibromoisocyanuric acid (DBICA), and tetrabutylammonium tribromide among others. The treatment may occur in the presence of mild catalysts or additives including but not limited to common acids (e.g., hydrochloric acid, acetic acid, p-toluenesulfonic acid, trifluoroacetic acid, etc.) or bases (e.g., sodium bicarbonate, sodium hydroxide, amines) to produce products as described in Reaction A. This may be accomplished using a variety of common benign solvents (water, tetrahydrofuran, methanol, acetonitrile . . . ) and may also be accomplished without need for protection from moisture or inert atmosphere. For said treatments, proposed temperature ranges include −78° C. to the reflux point of the chosen solvent (˜150° C.).

Aspects of the technology include using the halogenated resorcinol's of Reaction A (“Resorcinol A”) described above as scaffolding for the synthesis of other compounds as further described below.

### II. Modification at 2-Position of 5-Functionalized Resorcinols

Halogenated resorcinol groups may serve as a stable synthetic intermediate that may be used as a substrate for the synthesis of both known and unknown cannabinoids. For example, the halogenated resorcinols described above may be used as substrates.

Accordingly, aspects of the technology include adding nucleophiles at the 2-position for certain resorcinols. In particular, a resorcinol selected from the following group:

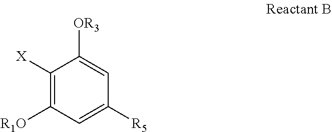

may be treated with a metallating species such as zinc)(Zn⁰), a lower alkyllithium (e.g., e.g., n-butyllithium or t-butyllithium), or magnesium)(Mg⁰), and reacted with an electrophilic metal species, such as boronyl chlorides (ClB(OR)₂), stannyl chlorides (ClSn(R)₃), and silyl chlorides (ClSi(R)₃). The expression, “lower alkyl,” as used herein, refers to a C1-C8 alkyl, which may be linear or branched, and which may include a double bond, e.g., an allyl. In some instances, reactant B may be treated with a palladium source and reacted in a cross coupling with a cross coupling viable, metal source such as bis(pinacoloto)borane (B(pin)₂) or hexamethylditin ((SnMe₃)₂) to form a 2-metallated resorcinol, where [M] is one of B(OR)₂, SnR₃, or SiR₃ having the following structure:

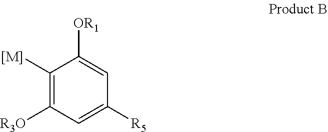

Thus, aspects of the technology described herein is:

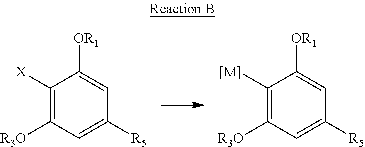

Resorcinols may be selected with particular functional groups at R1, R3, and R5 for applications. For synthesis of certain cannabinoids (e.g., cannabidiol), a resorcinol may be selected with the desired functional group (e.g., n-pentyl). In other instances, synthesis of certain cannabinoids and cannabinoid derivatives may include other intermediate steps where it may be desirous to have other functional groups may at R1, R3, and R5.

As such, in Reaction B, X may be chlorine, bromine, iodine, acetate, triflate or any other useful functional group. In aspects, R1 and R3 each may be one of H, acetate or other esters, a lower alkyl (e.g., methyl), benzyl, or other ethers (e.g., methoxymethyl (MOM)), a lower alkyl carbonate, a silane protecting group (e.g., a lower alkyl silane), or any other useful functional group. In aspects of the technology, R5 may be a lower alkyl group (e.g., ethyl, propyl, butyl, pentyl, allyl . . . ), a phenyl, a substituted phenyl, a lower alkenyl (e.g., a vinyl, a substituted vinyl), or a lower alkynyl. As used herein, the expression “lower alkenyl” refers to C2-C8 alkenyl, and the expression “lower alkynyl” refers to a C2-C8 alkynyl. It is understood that the sp² carbon of the lower alkenyl and sp carbon of the lower alkynyl is bound directly to the C5-position of the resorcinol.

The addition of metal species at the 2-position described above may be accomplished by treatment of Reactant B with di-metal species such as bis(pinacoloto)borane, hexabutylditin in the presence of a suitable catalyst, including palladium, nickel, copper, gold, silver, iron, or cobalt, Pd(ddpf)₂Cl₂, Pd(PPh₃)₂Cl₂, Pd(PPh₃)₄, Ni(cod)₂, NiI₂, NiBr₂, NiCl₂, and Ni(acac)₂. Any suitable ligand/base/additive may be used with the above metalation reactions, including, but not limited to pyridines, bipyridines, phenanthrolines, terpyridines, bisoxazoline, pyridine bisoxazoline, phosphines, metal halide salts (sodium iodide, sodium fluoride, magnesium chloride etc.), metal alkoxide salts (lithium methoxide, sodium methoxide, etc.), amines (triethylamine, diisopropylethylamine, etc.), carbonates (potassium carbonate, cesium carbonate, sodium carbonate, lithium carbonate, etc.), to afford the corresponding cross-coupling viable metal species.

Additionally, any suitable solvent may be used with the above-described metalation reactions, including dimethylformamide (DMF), dimethylacetamide, and other amide solvents, tetrahydrofuran (THF) and other ethereal solvents, toluene and other aromatic solvents, dichloromethane and other halogenated solvents, acetonitrile, dimethylsulfoxide, hydrocarbon solvents, methanol and other alcohol solvents, etc.

In aspects of the technology, reaction times may be from one to twenty-four hours and temperatures may range from about −78 to about 100° C.

Additionally, conversion from the halide to an organometallic may be performed. In such a conversion a halide (X) may be substituted with lithium, copper, magnesium, or zinc metal to form a reactive organometallic intermediate. These intermediates may be used in corresponding cross-coupling reactions (Negishi reactions, Kumada reactions, etc.) or directly treated with an electrophile such as citral, geranyl bromide, or verbenol acetate in any viable solvent, including toluene and other aromatic solvents, tetrahydrofuran and other ethereal solvents, DMSO, hydrocarbon solvents, etc. Reactions times may be between 0 and 24 hours and temperatures may range from −78 to 100° C.

The treatment may occur in the presence of mild catalysts or additives including but not limited to common acids (hydrochloric acid, acetic acid, p-toluenesulfonic acid, trifluoroacetic acid, etc . . . ) or bases (sodium bicarbonate, sodium hydroxide, amines) to produce products as described in Reaction A and Reaction B. This may be accomplished using a variety of common benign solvents (water, tetrahydrofuran, methanol, acetonitrile . . . ) and may also be accomplished without need for protection from moisture or inert atmosphere. For said treatments, proposes temperature ranges include −78° C. to the reflux point of the chosen solvent (˜100° C.).

Aspects of the technology include using the resorcinols of Reaction C (“Resorcinol C”) and Reaction D (“Resorcinol D”) described above as scaffolding for the synthesis of cannabinoids as further described below.

### III. Substitution at 1,3-Position 2 Halogenated, 5-Functionalized Resorcinols

Where halogenated or metalized resorcinol groups have a hydroxide at the 1 and 3 position, one or both of the hydroxides may be substituted with different functional groups. For example, the different functional group may serve as protecting groups during other reactions.

Accordingly, aspects of the technology include modification at the 1-position and/or 3-position for certain resorcinols. In particular, a resorcinol of the following structure

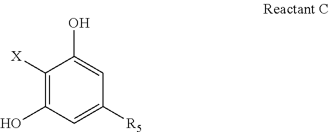

may be reacted with a suitable base, such as sodium bicarbonate, potassium carbonate, triethylamine, or dimethylamino pyridine in a suitable solvent such as DMF, THF, or dichloromethane. The resulting mixture may then be treated with a corresponding halogenated precursor such as methyl iodide, benzyl bromide, trimethylsilyl chloride, t-butyldimethylsilyl chloride, SEM chloride, or acetyl chloride. In some instances, the protecting group precursor may not contain a halogen, such as in the case of acetic anhydride. In some instances, a protecting group may not require a base for the substitution reaction, such as the case of protection with a tetrahydropyranyl (THP) group, where an acid may be desired to produce

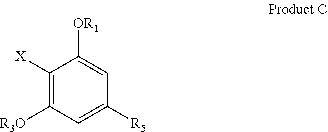

Thus, aspects of the technology described herein is:

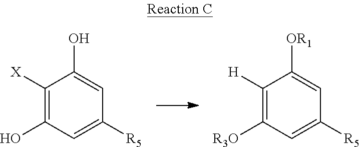

Resorcinols may be selected with particular functional groups at R1, R3, and R5 for applications. For instance, the synthesis of certain cannabinoids (e.g., cannabidiol), a resorcinol may be selected with the desired functional groups (e.g., n-pentyl) at R5. In other instances, synthesis of certain cannabinoids and cannabinoid derivatives may include other intermediate steps where it may be desirous to have other functional groups at R1, R3, and R5. As such, in Reaction C, X may be chlorine, any boron group, bromine, iodine, acetate, triflate, any alkyl stannane, any alkyl silane or any other useful functional group. In one aspect, R1 and R3 may each may be one of H, a lower alkyl ester, a lower alkyl, benzyl or other ethers, a lower alkyl carbonate, a silane protecting group (e.g., a lower alkyl silane), or any other useful functional group. In aspects of the technology, R5 may be an alkyl group (ethyl, propyl, butyl, pentyl, allyl, etc.), a phenyl, a substituted phenyl, a lower alkenyl (e.g., a vinyl, a substituted vinyl), or a lower alkynyl, with the proviso that the sp2 carbon of the lower alkenyl and sp carbon of the lower alkynyl is bound directly to the C5-position of the resorcinol.

The substitution at the 1-position and/or 3 position described above may be accomplished by treatment of Reactant C with a suitable base, such as sodium bicarbonate, potassium carbonate, triethylamine or any trialkylamine or dimethylamino pyridine and optionally any suitable acid or base catalyst or additive such as dimethylamino pyridine, in a suitable solvent such as DMF, THF, or dichloromethane. The resulting mixture can then be treated with a corresponding halogenated precursor such as methyl iodide, benzyl bromide, trimethylsilyl chloride, t-butyldimethylsilyl chloride, 2-(trimethylsilyl)ethoxymethyl (SEM) chloride, methoxy methyl (MOM) chloride, or acetyl chloride.

In some instances, the protecting group precursor may not contain a halogen, such as in the case of acetic anhydride. In some instances, a protecting group may not require a base for the substitution reaction, such as the case of protection with a THP group, where an acid may be desired. Any suitable base/additive may be used with the above substitution reactions, including, but not limited to metal halide salts (sodium iodide, sodium fluoride, magnesium chloride etc.), metal alkoxide salts (lithium methoxide, sodium methoxide, etc.), amines (triethylamine, diisopropylethylamine, etc.), carbonates (potassium carbonate, cesium carbonate, sodium carbonate, lithium carbonate, etc.), to afford the corresponding resorcinol.

Additionally, any viable solvent may be used with the above-described reactions, including dimethylformamide, dimethylacetamide, and other amide solvents, tetrahydrofuran and other ethereal solvents, toluene and other aromatic solvents, dichloromethane and other halogenated solvents, acetonitrile, dimethylsulfoxide, hydrocarbon solvents, methanol and other alcohol solvents, etc.

In aspects of the technology, reaction times may be from one to twenty-four hours and temperatures may range from about −78 to about 100° C. The treatment may occur in the presence of mild catalysts or additives including but not limited to common acids (hydrochloric acid, acetic acid, p-toluenesulfonic acid, trifluoroacetic acid, etc . . . ) or bases (sodium bicarbonate, sodium hydroxide, amines) to produce products as described in Reaction C. This may be accomplished using a variety of common benign solvents (water, tetrahydrofuran, methanol, acetonitrile . . . ) and may also be accomplished without need for protection from moisture or inert atmosphere.

### IV. Examples

Each compound described herein was characterized by 1H-NMR. The NMR spectral data are consistent with the depicted compounds.

### a) Example 1: Synthesis of 2-Iodo-5-pentyl-resorcinol Using MTBE-H₂O

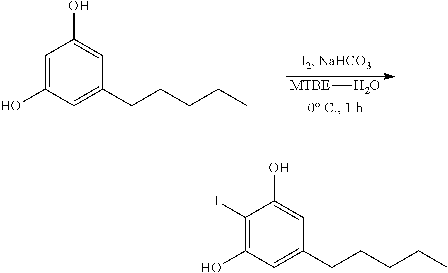

In a first example, Olivetol (1 g, 5.55 mmol) and sodium bicarbonate (466 mg, 16.7 mmol) were dissolved in a solution of methyl t-butyl ether (2.2 mL) and H₂O (7.4 mL). The mixture was cooled to 0° C. and a solution of iodine (2.8 g, 11.1 mmol) in methyl t-butyl ether (5.3 mL) was added dropwise. The reaction mixture was stirred at 0° C. for 1 h and was subsequently diluted with methyl t-butyl ether (4.4 mL). A solution of sodium sulfite (466 mg, 11.1 mmol) in water (4.4 mL) as added slowly. The mixture was allowed to warm to room temperature and stirred for 30 min. The mixture was extracted with diethyl ether (3×50 mL) and the combined organic extracts were dried (MgSO₄), filtered and concentrated in vacuo. The product was obtained as a beige solid (1.56 g, 92%) without further purification. Additionally/alternatively, the product may be recrystallized from heptane or pentane.

### b) Example 2: Synthesis of 2-Iodo-5-pentyl-resorcinol Using THF-H₂O

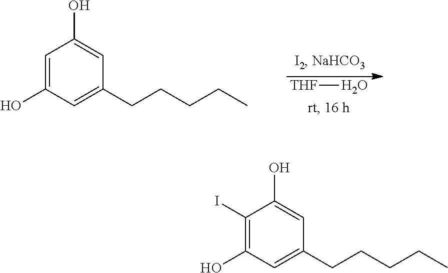

In a second example, Olivetol (1 equiv.) was dissolved in a mixture of THF-H₂O (1:1, 0.5 M) in a foil wrapped reaction vessel. Iodine (1 equiv.) was added followed by sodium bicarbonate (1 equiv.) slowly added in portions and the reaction was allowed to stir at room temperature overnight. The reaction was quenched by the addition of sodium thiosulfate and diluted with ethyl acetate. The layers were separated, the aqueous layer was extracted with ethyl acetate and the combined organic layers were washed with brine, dried (sodium sulfate), filtered through a plug of silica and concentrated in vacuo. An orange solid was obtained, taken up in pentane and cooled to −20° C. to afford 2-iodo-5-pentyl-resorcinol as white needlelike crystals.

### c) Example 3: Synthesis of 2-Iodo-5-pentyl-resorcinol Using CH₃CN

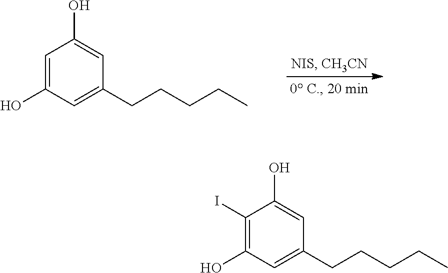

In a third example, to a vial charged with stir bar was added a solution of olivetol (100 mg, 0.555 mmol) in acetonitrile (1 mL). The vial was sealed and cooled to 0° C. A solution of N-iodosuccinimide (124.9 mg, 0.555 mmol) in acetonitrile (1 mL) was added dropwise. The reaction was stirred at 0° C. for 20 minutes before washing with dichloromethane (3×). The combined organic extracts were washed with brine and diluted in heptane. The organic extracts were then dried (MgSO₄), filtered through a pad of celite, and concentrated in vacuo to afford a light orange oil. The reaction was taken up in warm heptane (55° C.) and cooled to −20° C. A white powder precipitated after 16 h. The white powder was filtered and washed with cold heptane to afford 2-iodo-5-pentyl-resorcinol (64 mg, 38%) as a white powder.

### d) Example 4: Synthesis of 1,3-methoxy-2-iodo-5-pentyl-resorcinol

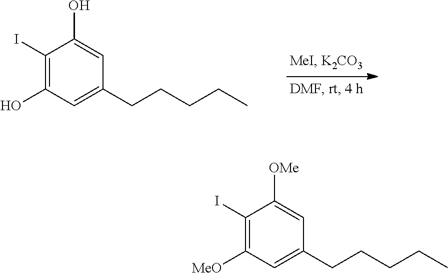 [SMILES: CCCCCc1cc(O)c(I)c(O)c1 ; CCCCCc1cc(OC)c(I)c(OC)c1]

In a fourth example, to an oven-dried, 250 mL round bottom flask was added DMF (81 mL) and 2-iodo-olivetol (5 g, 16.3 mmol). The solution was sparged with nitrogen gas for 10 minutes. Potassium carbonate (6.77 g, 49.0 mmol) was added in one portion under a nitrogen atmosphere. The mixture was stirred under nitrogen (1 atm) and a purple color was observed. Methyl iodide (6.98 g, 49.0 mmol) was added in one portion via syringe and the reaction was stirred for 3.75 h under nitrogen (1 atm). At this time, the reaction was deemed complete by TLC analysis (40:1 EtOAc/Heptane, 12 stain). The reaction was diluted with H₂O (100 mL) and extracted with a solution of petroleum ether/ether (2:1, 3×100 mL). The combined organic extracts were washed with brine, dried (MgSO₄), filtered through a pad of celite, and concentrated in vacuo to afford a crude yellow oil (5.75 g). The oil was purified by flash column chromatography (SiO₂, pet ether/ether) to afford the product as a hazy oil (5.08 g, 93%).

### e) Example 5: Synthesis of 1,3-acetoxy-2-iodo-5-pentyl-resorcinol

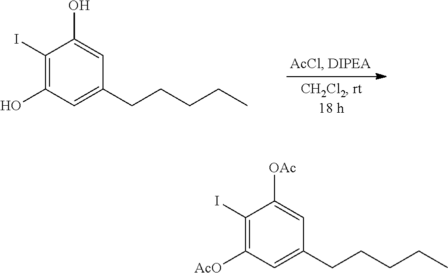 [SMILES: CCCCCc1cc(O)c(I)c(O)c1 ; CCCCCc1cc(OC(C)=O)c(I)c(OC(C)=O)c1]

In a fifth example, to an oven-dried 20 mL vial charged with stir bar and purged with nitrogen gas was added 2-iodo-olivetol (500 mg, 1.63 mmol) and dichloromethane (5.4 mL). The mixture was cooled to 0° C. and DIPEA (443 g, 3.43 mmol) was added in one portion via syringe while mixture was stirred. A purple color was observed. The mixture was stirred for 5 min at 0° C. Acetyl chloride (385 mg, 4.90 mmol) was added dropwise via syringe. The reaction rapidly changed from purple to a clear yellow-orange color. The reaction was allowed to warm to room temperature and stirred for 18 h. The reaction was concentrated under a stream of nitrogen and diluted with petroleum ether (10 mL). The solution was filtered and concentrated in vacuo to afford an orange oil. The oil purified by flash column chromatography (SiO₂, pet ether/ether) to afford the product as a colorless oil (540 mg, 85%).

### f) Example 6: Synthesis of 1,3-methoxy-2-pinacolboronyl-5-pentyl-resorcinol Using THF

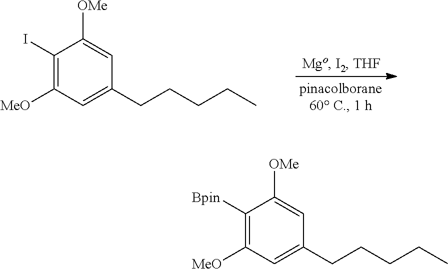

In a sixth example, magnesium (72.7 mg, 2.99 mmol) and iodine (19 mg, 0.075 mmol) were charged into a hot vial and cooled under a stream of nitrogen. The solids were suspended in THF (0.5 mL) to give an orange-brown suspension. Pinacol borane (383 mg, 2.99 mmol) was added via syringe. 1,3-methoxy-2-iodo-5-pentyl-resorcinol (500 mg, 1.49 mmol) as a solution in THF (0.5 mL) was added dropwise via syringe, followed by a THF (0.5 mL) wash. The vial was heated to 60° C. and stirred for 1 h. The reaction was cooled to 0° C., diluted with petroleum ether, quenched with 2 N HCl (2 mL), and stirred for 15 min. The organic layer was separated and the aqueous layer was extracted with petroleum ether (3×4 mL). The combined organic layers were dried (MgSO4), filtered, and concentrated in vacuo. The residue was purified by column chromatography (SiO2, pet ether/ether) to afford the product as a colorless oil.

### h) Example 8: Synthesis of 1,3-trimethylsiloxy-2-iodo-5-pentyl-resorcinol

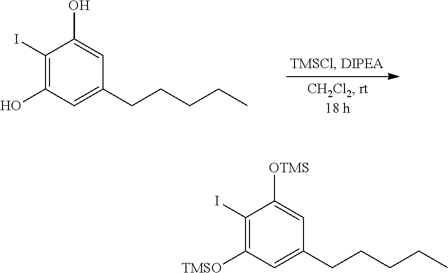

In an eight example, to a vial charged with stir bar was added 2-iodo-olivetol (500 mg, 1.63 mmol) and the vial was sealed. The 2-iodo-olivetol was dissolved in dichloromethane (5.44 mL) and DIPEA (422 mg, 3.27 mmol) was added via syringe. The vial was cooled to 0° C. and trimethylsilyl chloride (532 mg, 4.90 mmol) was added dropwise with rapid stirring. The reaction was allowed to warm to room temperature for 18 h. At this time, an additional equivalent of trimethylsilyl chloride and DIPEA were added and the reaction was stirred an additional 24 h. The reaction was concentrated under nitrogen and diluted with petroleum ether, filtered, washed with sat. aq. sodium bicarbonate, 0.1 M HCl and brine. The organic extract was dried (MgSO₄), filtered, and concentrated in vacuo to afford a crude orange oil. The crude oil was purified by column chromatography (SiO₂, pet ether/ether) to afford the product (348 mg, 47%) as a clear oil.

### i) Example 9: Synthesis of 1,3-[2-(trimethylsilyl)ethoxy]methyl acetoxy-2-iodo-5-pentyl-resorcinol

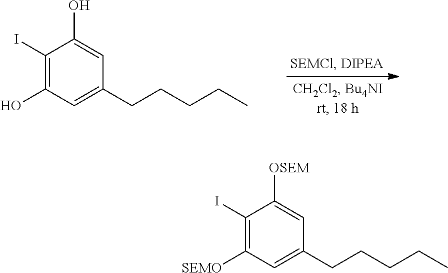

In a ninth example, to a vial charged with stir bar was added 2-iodo-olivetol (500 mg, 1.63 mmol) and Bu4NI (60 mg, 0.16 mmol) and the vial was sealed. The solids were dissolved in dichloromethane (5.44 mL) and DIPEA (654 mg, 5.06 mmol) was added via syringe. The reaction cooled to 0° C. and SEM-Cl (817 mg, 4.90 mmol) was added via syringe with stirring. The reaction was allowed to warm to room temperature and stir for 18 h. At this time, the reaction was deemed complete by TLC analysis and was concentrated under nitrogen, diluted with petroleum ether, filtered and concentrated in vacuo to afford a crude orange oil. The crude oil was purified by column chromatography (SiO2, pet ether/ether) to afford the product (748 mg, 81%) as a milky oil.

### j) Example 10: Synthesis of 1,3-benzyloxy-2-iodo-5-pentyl-resorcinol

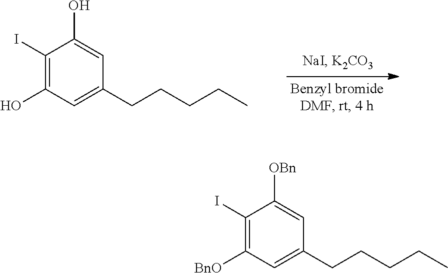 [SMILES: CCCCCc1cc(O)c(I)c(O)c1 ; CCCCCc1cc(OCc2ccccc2)c(I)c(OCc2ccccc2)c1]

In a tenth example, to a dry 11 dram vial charged with stir bar was added sodium iodide (1.47 g, 9.80 mmol) and potassium carbonate (1.35 g, 9.80 mmol). The vial was sealed and purged with nitrogen. Benzyl bromide (1.68 g, 9.80 mmol) was added in one portion via syringe. A solution of 2-iodo-olivetol (1.00 g, 3.27 mmol) in acetone (5.4 mL) was added via syringe with rapid stirring. The reaction was heated to 55° C. A rapid color change from pale yellow to dark red was observed. After 18 h, the reaction was quenched by addition of methanol (1 mL) and additional potassium carbonate (450 mg) and stirred for 15 min at 55° C. The reaction was filtered through celite and concentrated in vacuo. The reaction was diluted with pet ether and the solids were removed by filtration. The solution still contained benzyl bromide as deemed by TLC analysis and triethylamine (0.56 mL) was added. After 15 min, another aliquot of triethylamine (0.56 mL) was added and the mixture was stirred. The hazy solution was washed with 2 N HCl (4 mL), 1 N NaOH (4 mL), brine (4 mL), and dried (MgSO₄). The mixture was filtered and concentrated in vacuo. The residue was purified by column chromatography (SiO₂, pet ether/ether) to afford the product.

### m) Example 11: Synthesis of 1,3-benzyloxy-2-pinacolboronyl-5-pentyl-resorcinol

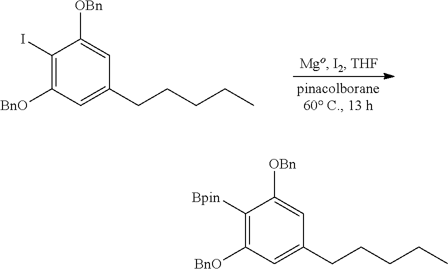

In an eleventh example, magnesium (24.3 mg, 0.43 mmol) and iodine (19 mg, 0.075 mmol) were charged into a hot vial and cooled under a stream of nitrogen. The solids were suspended in THF (0.1 mL) to give an orange-brown suspension. Pinacol borane (83.6 mg, 0.65 mmol) was added via syringe. 1,3-benzyloxy-2-iodo-5-pentyl-resorcinol (159 mg, 0.33 mmol) as a solution in THF (0.4 mL) was added dropwise via syringe. The vial was heated to 60° C. and stirred for 13 h. The reaction was quenched with 0.1 M HCl (0.5 mL) (after being diluted with pet ether) and stirred for 30 min. The organic layer was separated and the aqueous layer was extracted with petroleum ether (3 mL). The combined organic layers were dried (MgSO4), filtered, and concentrated in vacuo. The residue was purified by column chromatography (SiO₂, pet ether/ether) to afford the product (87 mg) as a colorless oil.

### n) Example 12: Synthesis of 1,3-[2-(trimethylsilyl)ethoxy]methyl acetoxy-2-pinacolboronyl-5-pentyl-resorcinol

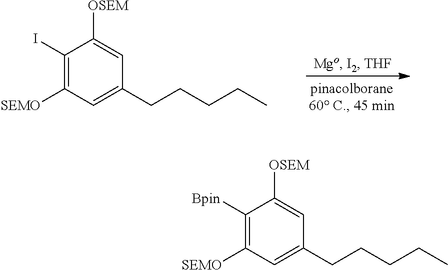

In a twelfth example, magnesium (24.3 mg, 0.43 mmol) was charged into an oven dried vial and cooled under a stream of nitrogen. The solids were suspended in THF (0.1 mL) to give an orange-brown suspension. Pinacol borane (83.6 mg, 0.65 mmol) was added via syringe. 1,3-SEM-2-iodo-5-pentyl-resorcinol (185 mg, 0.33 mmol) as a solution in THF (0.4 mL) was added dropwise via syringe. The vial was heated to 60° C. and stirred for 45 min. The reaction was quenched with 0.1 M HCl (0.5 mL) (after being diluted with pet ether) and stirred for 30 min. The organic layer was separated and the aqueous layer was extracted with petroleum ether (3 mL). The combined organic layers were dried (MgSO4), filtered, and concentrated in vacuo. The residue was purified by column chromatography (SiO2, pet ether/ether) to afford the product (82 mg) as a colorless oil.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In other words, functional elements being performed by a single or multiple components and individual functions can be distributed among different components. In this regard, any number of the features of the different embodiments described herein may be combined into one single embodiment and alternate embodiments having fewer than or more than all of the features herein described as possible.

While various embodiments have been described for purposes of this disclosure, various changes and modifications may be made which are well within the scope of the disclosed methods. Numerous other changes may be made which will readily suggest themselves to those skilled in the art and which are encompassed in the spirit of the disclosure.

1. A compound having the following structure: 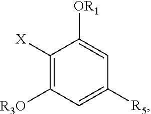
wherein X is selected from the group consisting of I, bis(pinacolato)diboron (Bpin), B(OH)₂, B(OR₆)₂, Br, Sn(R₇)₃, Si(Me)₃, Si(R₈)₃, OTf, Cl, Mg(II)I, Zn(II)I, cuprate, lithium, Mg(II)Br, and Zn(II)Br,
wherein each of R₁ and R₃ is selected from the group consisting of THP, Benzyl, 2-(trimethylsilyl)ethoxymethyl (SEM), and a silane protecting group;
wherein R₅ is selected from the group consisting of a lower alkyl group, a phenyl, a substituted phenyl, a lower alkenyl, and a lower alkynyl.

2. The compound of claim 1, wherein R₁ and R₃ are different.

3. The compound of claim 1, wherein each of R₆, R₇, and R₈, is selected from the group consisting of a lower alkyl group, a phenyl, a substituted phenyl, a lower alkenyl, and a lower alkynyl.

4. A compound having the following structure: 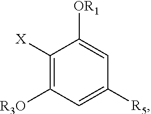
wherein X is selected from the group consisting of bis(pinacolato)diboron (Bpin), B(OH)₂, B(OR₆)₂, Br, Sn(R₇)₃, Si(Me)₃, Si(R₈)₃, OTf, Mg(II)I, Zn(II)I, a cuprate, lithium, Mg(II)Br, and Zn(II)Br,
wherein each of R₁ and R₃ is selected from the group consisting of hydrogen and acetate;
wherein R₅ is a functional group selected from the group consisting of a lower alkyl group, a phenyl, a substituted phenyl, a lower alkenyl, and a lower alkynyl.

5. The compound of claim 4, wherein R₁ and R₃ are different.

6. The compound of claim 4, wherein each of R₆, R₇, and R₈, is selected from the group consisting of a lower alkyl group, a phenyl, a substituted phenyl, a lower alkenyl, and a lower alkynyl.

7. A compound having the following structure: 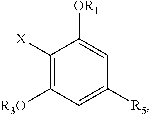
wherein X is selected from the group consisting of B(OR₆)₂, Sn(R₇)₃, Si(R₈)₃, OTf, Cl, Mg(II)I, Zn(II)I, a cuprate, and Zn(II)Br;
wherein each of R₁ and R₃ is selected from the group consisting of methyl and methoxymethyl (MOM);
wherein R₅ is a functional group selected from the group consisting of a lower alkyl group, a phenyl, a substituted phenyl, a lower alkenyl, and a lower alkynyl.

8. The compound of claim 7, wherein R₁ and R₃ are different.

9. The compound of claim 7, wherein each of R₆, R₇, and R₈, is selected from the group consisting of a lower alkyl group, a phenyl, a substituted phenyl, a lower alkenyl, and a lower alkynyl.

10. A compound having the following structure: 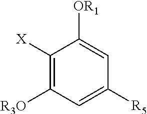
wherein X is selected from the group consisting of Bpin, B(OH)₂ and lithium,
wherein R₁ and R₃ each is MOM;
wherein R₅ is selected from the group consisting of a lower alkyl group, a phenyl, a substituted phenyl, a lower alkenyl, and a lower alkynyl.

11. The compound of claim 10, wherein R₁ and R₃ are different.

12. A compound having the following structure: 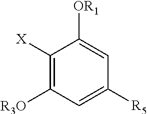
wherein X is selected from the group consisting of Bpin, B(OH)₂, Si(Me)₃, and lithium,
wherein each of R₁ and R₃ is MOM;
wherein R₅ is selected from the group consisting of a lower alkyl group, a phenyl, a substituted phenyl, a lower alkenyl, and a lower alkynyl.

13. A compound having the following structure: 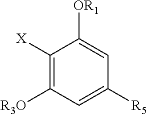
wherein X is selected from the group consisting of Mg(II)Br and a cuprate;
wherein each of R₁ and R₃ is methyl; and
wherein R₅ is selected from the group consisting of a lower alkyl group, a phenyl, a substituted phenyl, a lower alkenyl, and a lower alkynyl.

14. A compound having the following structure: 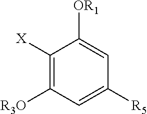
wherein X is Cl;
wherein each of R₁ and R₃ is acetate; and
wherein R₅ is selected from the group consisting of a lower alkyl group, a phenyl, a substituted phenyl, a lower alkenyl, and a lower alkynyl.

15. A method of halogenating a resorcinol, the method comprising:
providing a first compound having the following structure: 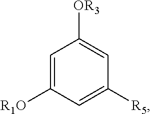
wherein R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and further wherein R₅ is selected from the group consisting of a lower alkyl group, a phenyl, a substituted phenyl, a lower alkenyl, and a lower alkynyl; and
treating the compound with a halogenating agent in the presence of a solvent, wherein the halogenating agent is selected from the group consisting of bromine (Br₂), iodine (I₂), N-chlorosuccinimide (NCS), N-bromosuccinimide (NBS), N-iodosuccinimide (NIS), 1,3-dichloro-5,5-dimethylhydantoin (DCDMH), 1,3-dibromo-5,5-dimethylhydantoin (DBDMH), trichloroisocyanuric acid (TCICA), dibromoisocyanuric acid (DBICA), and tetrabutylammonium tribromide.

16. The method of claim 15: further comprising
adding a catalyst, wherein the catalyst is selected from the group consisting of hydrochloric acid, acetic acid, p-toluenesulfonic acid, trifluoroacetic acid, sodium bicarbonate, sodium hydroxide, an amine, and a combination thereof

17. The method of claim 15, wherein the solvent is selected from the group consisting of water, tetrahydrofuran, methanol, acetonitrile, methyl t-butyl ether and a combination thereof.

18. A method of modifying a resorcinol comprising:
providing the resorcinol having the following structure: 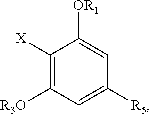
wherein x is a halogen, further wherein R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and further wherein R₅ is selected from the group consisting of a lower alkyl group, a phenyl, a substituted phenyl, a lower alkenyl, and a lower alkynyl;
treating the resorcinol with bis(pinacoloto)borane or hexabutylditin in the presence of a suitable catalyst, comprising palladium, nickel, copper, gold, silver, iron, or cobalt, Pd(ddpf)₂Cl₂, Pd(PPh₃)₂Cl₂, Pd(PPh₃)₄, Ni(cod)₂, NiI₂, NiBr₂, NiCl₂, and Ni(acac)₂, or a combination thereof, in the presence of a base selected from the group consisting of a pyridine, a bipyridine, a phenanthroline, a terpyridine, a bisoxazoline, pyridine bisoxazoline, a phosphine, a metal halide salt, a metal alkoxide salt, an amine, a carbonate, and a combination thereof.

19. The method of claim 18, wherein X is selected from the group consisting of chlorine, bromine, iodine, acetate, and triflate.

20. A method of modifying a resorcinol comprising:
providing the resorcinol having the following structure: 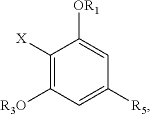
wherein X is a halogen or a metal;
each of R₁ and R₃ is hydrogen;
treating the resorcinol with a base selected from the group consisting of sodium bicarbonate, potassium carbonate, triethylamine, dimethylamino pyridine, and a combination thereof,
in the presence of a solvent selected from the group consisting of as DMF, THF, and dichloromethane; and
treating the mixture with a halogenating agent selected from the group consisting of methyl iodide, benzyl bromide, trimethylsilyl chloride, t-butyldimethylsilyl chloride, SEM chloride, and acetyl chloride.

21. A method of modifying a resorcinol comprising:
providing the resorcinol having the following structure: 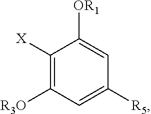
wherein x is a halogen, further wherein R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and further wherein R₅ is selected from the group consisting of a lower alkyl group, a phenyl, a substituted phenyl, a lower alkenyl, and a lower alkynyl;
treating the resorcinol with a metallating species to form a treated resorcinol; and
reacting the treated resorcinol with electrophilic metal species in the presence of a solvent.

22. The method of claim 21, wherein the metallating species is selected from the group consisting of zinc, lower alkyllithium, and magnesium.

23. The method of claim 21, wherein the electrophilic metal species is selected from the group consisting of boronyl chlorides, stannyl chlorides, pinacol borane, and silyl chlorides.

24. The method of claim 21, wherein the solvent is selected from the group consisting of dimethylformamide (DMF), dimethylacetamide, tetrahydrofuran (THF), toluene, dichloromethane, acetonitrile, dimethylsulfoxide, hydrocarbon solvents and a combination thereof.

25. A method of modifying a resorcinol comprising:
providing the resorcinol having the following structure: 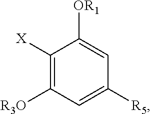
wherein x is a halogen, and further wherein R₁ and R₃ each are selected from the group consisting of hydrogen, acetate, a lower alkyl ester, a lower alkyl, benzyl, a lower alkyloxy-lower alkyl, a lower alkyl carbonate, a silane protecting group, and further wherein R₅ is selected from the group consisting of a lower alkyl group, a phenyl, a substituted phenyl, a lower alkenyl, and a lower alkynyl;
treating the resorcinol with a di-metal species to form a treated resorcinol; and
reacting the treated resorcinol with electrophilic metal species in the presence of a solvent.

26. The method of claim 25, where in the di-metal species is selected from the group consisting of bis(pinacoloto)borane, hexabutylditin in the presence of a suitable catalyst, including palladium, nickel, copper, gold, silver, iron, or cobalt, Pd(ddpf)₂Cl₂, Pd(PPh₃)₂Cl₂, Pd(PPh₃)₄, Ni(cod)₂, NiI₂, NiBr₂, NiCl₂, and Ni(acac)₂.

27. The method of claim 25, wherein the solvent is selected from the group consisting of dimethylformamide (DMF), dimethylacetamide, tetrahydrofuran (THF), toluene, dichloromethane, acetonitrile, dimethylsulfoxide, hydrocarbon solvents and a combination thereof.
